(19) United States
(12) Patent Application Publication
(10) Pub. No.: US 2005/0222250 A1
(43) Pub. Date: Oct. 6, 2005

(54) Curcumin for the prevention and/or treatment of tissue damage

(76) Inventor: Mohiaddin Rezvani, Oxford (GB)

Correspondence Address: Eitan Pearl Latzer & Cohen Zedek, Suite 1001, 10 Rockefeller Plaza, New York, NY, 10020, US

(21) Appl. No.: 10/510,981

(22) PCT Filed: Apr. 16, 2003
(86) PCT No.: PCT/GB03/01694

§ 371 Date: May 19, 2005

(30) Foreign Application Priority Data
Apr. 16, 2002 (GB) 0208691.6
Aug. 8, 2002 (GB) 0218412.5

(51) Int. Cl. A61K 31/34; A61K 31/12

(52) U.S. Cl. 514/461; 514/685

(57) ABSTRACT

A combination of curcumin, an antioxidant, especially α-tocopherol, and at least one edible oil, especially sunflower oil, is useful in the prevention and/or treatment of tissue damage caused by non-physical insult, especially mucositis or CNS damage caused by cancer therapy

The present invention relates to biological response modifiers for use in preventing and treating tissue damage.

Approximately 50% of all cancer patients receive radiotherapy at one stage in their course of treatment. Local tumour control is directly related to the radiation dose. The greater the radiation dose, the greater the probability of tumour control. Although it may be possible, in theory, to eradicate a localised tumour with a large dose of radiation, in practice the damage to normal tissues, which is also dose-dependent, is a major limiting factor. Therefore, the dose delivered to the tumour is always compromised for the danger of damaging normal tissues adjacent the tumour.

Advances in the administration of radiation such as dose fractionation, low dose-rate irradiation, application of radiosensitisers or radioprotectors, and better localisation techniques, have increased the therapeutic gain. However, in reality, even, with limited doses of radiation, there are always relatively sensitive patients who may develop abnormal tissue reactions. Therefore, normal tissue damage is almost an inevitable consequence of radiotherapy in the majority of cases. For example, in radiotherapy of laryngopharyngeal tumours, 95% of the patients may experience a kind of early transient reaction, and 61% suffer more persistent late reactions [Rezvani, et al., Brt. J. Radiol. (1991), 64:1122-1133].

The stem cells of the epithelial lining of the oral mucosa are non-specifically affected by many anti-cancer agents including radiation. Mucositis induced by chemotherapy or radiotherapy is an important dose-limiting side effect of cancer therapy [Sonis, S. T., Oral Oncol. (1998);34:39-43]. Radiation-induced mucositis of the upper aerodigestive tract, in particular, is a major dose-limiting factor in the treatment of head and neck tumours. Apart from being a painful and distressing experience, radiation-induced mucositis, coupled with the associated xerostomia, may lead to poor oral hygiene and weight loss in head and neck cancer patients. A planned course of treatment may often be interrupted to allow for the healing of this acute reaction, and may impair the outcome of treatment.

In one study, mucosal reactions were seen in almost all patients (95%) treated for head and neck cancer with 68% of the patients having ulceration or fibrinous reaction of the mucosal membrane. In another study severe acute mucosal effects were reported in 52% of the patients treated with radiotherapy for carcinoma of the oral cavity and the oropharynx. Mucositis occurs in approximately 40% of cancer patients treated with chemotherapy with 50% of them requiring modification of their cancer treatment and/or analgesia.

A number of mouth washes containing antiseptic and/or analgesics have been used in the treatment of radiation-induced mucositis. These treatment approaches are ineffective in preventing the development of mucositis and have also been shown to be detrimental in some cases [Foote, et al., J. Clin. Oncol. (1994), 12:2630-3]. Recently, a variety of novel therapeutic agents have been developed with limited success [Spadinger, et al., J. Clin. Oncol. (1994), 12: 1917-1922; Troussard, et al., Br. J. Haematol. (1995), 89:191-5; Farrell, et al., Int. J. Radiat Biol. (1999), 75: 609-620] with the exception of keratinocyte growth factor where promising results have been reported [Dörr, et al., Int J. Radiat Biol. (2001); 77: 341-347]. At present there is no effective method of treating radiation-induced mucositis.

The basis for the classical management of therapy-induced mucositis is pain relief, prevention of dehydration, providing adequate nutrition and controlling infection, such as candidiasis. A number of mouth washes containing antiseptic and/or analgesic agents have been developed and used but with no beneficial effects. This includes chlorhexidine and nystatin, glycerine, thymol, glycerine, lemon and hydrogen peroxide. These classical treatment approaches not only are inefficient in preventing the development of mucositis but have been shown to be detrimental in some cases. This includes the application of sodium bicarbonate and chlorhexidine. It appears that frequent mechanical cleansing of the mouth by sophisticated mouthwashes is harmful, as noted above, and that a simple saline solution is indicated as the most effective mouth wash in therapy-induced oral mucositis. However, damage to the parotid glands and disturbances in intercellular signalling after irradiation can result in a thickening of the saliva, a reduction in its production and dehydration of the mucus membrane. This can encourage infection which in turn will delay the healing of the ulceration. Based on this hypothesis some antibiotic based applications have proven to be beneficial in controlling mucositis.

The oral mucosa form a continuously renewing tissue consisting of a stratified squamous epithelium. Epithelial stem cells in the basal germinal layer proliferate, with a high rate of renewal, to balance the loss of cells from the surface layer. This rapid turnover of mucosal tissue renders this tissue responsive to both radiotherapy and chemotherapy. After irradiation while the cell loss continues from the superficial layers of the mucosa the deeper basal cells, killed or damaged by irradiation failing to produce replacements for the lost cells. Therefore, the mucosa become thinner and once the number of epithelial cells reach a critical level, broken mucosa develop as a result of the denudation, The protective effect of painting the mucosal surface with substances such as silver nitrate for several days before radiotherapy, Interleukuin-1, granulocyte colony-stimulating factor, granulocyte macrophage colony-stimulating factor and keratinocyte growth factor is possibly due to an increase in the number of mucosal cells (hyperplasia) prior to or during radiotherapy treatment.

An alternative approach in the treatment of treatment-induced mucositis is the use of substances that promote healing of the ulcerated mucosa and coating the mucus membrane to prevent further damage by the use of mucus binding substances. Sucralfate, which forms a barrier on the mucosa, has been shown to reduce the pain associated with mucositis. There are conflicting reports on the ability of sucralfate in prevention of mucositis [Epstein, et al., Oral Surg. Oral Med. Oral Pathol. 1992;73:682-9; Cengiz, et al., J. Clin. Gastroenterol. (1999), 28:40-43; Btiz, et al., Oral Oncol. (2000), 36:116-20].

Skin is an important tissue that is frequently exposed to radiation either accidentally or as a consequence of the treatment of cancer patients by radiotherapy. Acute radiation damage to the epidermis is related to the sterilisation of the reproductive component of the epidermis, stem cells. The full functional integrity of the skin can be preserved if there are sufficient surviving clonogenic cells within the basal layer, or within the shaft of hair follicles, to allow rapid repopulation of the surface. The absence of cell production results in the development of epithelial denudation (moist desquamation).

The spinal cord is one of the important dose-limiting normal tissues in radiotherapy. Excessive doses of radiation to the spinal cord can result in radiation myelopathy, a rare but serious complication of radiotherapy treatment for cancer. Latency times for radiation myelopathy in humans have been reported to vary between 4 months to 4 years after cervical orthoracic irradiation.

In experimental animals irradiation of the spinal cord leads to white matter necrosis within 3-8 months of irradiation. Radiation effects on the spinal cord have been extensively studied in a number of animal models. Factors such as the total radiation dose, overall treatment time, dose per fraction, dose-rate and the effects of changing the irradiated volume of the spinal cord have been examined as a guide to improving the therapeutic ratio in radiotherapy The majority of these studies have concentrated on optimising dose fractionation schedules.

Recently, attempts been made to modify this effect by the administration of therapeutic agents after irradiation [Hopewell, et al., New approaches to cancer therapy, unsaturated lipids and photodynamic therapy, Ed. D. F. Horrobin, pp 88-106. Churchill Communications, Burope (London), (1994); Homsey, et al., Int. J. Radiat. Oncol. Biol. Phys. 18, 1437-1442(1990)] or by transplantation of neural stem cells.

In classical radiobiology, the emphasis has been on identifying specific target cells responsible for the development of CNS injury. The CNS is a complex and extremely integrated tissue that depends greatly on cell to cell interactions. Radiation is a non-discriminating insult that probably damages all tissue components, including both endothelium and parenchyma. Therefore, it is unlikely that radiation injury would result from damaging a single target. Furthermore, the pattern of expression of a number of growth factors and cytokines is altered by radiation [Chiang, et al., Int. J. Radiation Oncology Biol. Phys. 24, 929-937 (1992)]. These are likely to have an effect on the development of delayed radiation injury in CNS.

Recently, Fike and Tofilon [Tofilon, et al., Rad. Res. 153: 357-370, (2000)] suggested that the production of reactive oxygen species (ROS), besides cell death, plays a major part in the development of radiation-induced myelopathies. This process is already recognised in the development of other CNS lesions such as those induced by trauma or ischaemia [Schwab, et al., Physiol. Rev. 76: 319-370, (1996)]. According to the hypothesis suggested by Tofilon & Fike (supra), radiation induced CNS injury is not the result of damage from a single instantaneous event but damage of a dynamic multifaceted nature which develops over time.

Radiosensitisers or radioprotectors modify the effects of radiation, but are required at the time of irradiation. While such substances can be used in radiotherapy, for example, their use is limited for post radiation treatment, and can not be used in the treatment of oversensitive patients who develop lesions as a side effect of radiotherapy, or for patients overexposed in radiation accidents.

Intervention treatments after radiotherapy and the application of various biological response modifiers have been suggested. Biological response modifiers are ideally substances that are administered after irradiation and, while these compounds could be of great value in the treatment of oversensitive or overexposed patients, they could also be used in cancer therapy in order to reduce the adverse effects of radiotherapy.

Ameliorating the risk of normal tissue damage would allow radiotherapists to apply larger doses of radiation, thereby to effect a more rapid cure, increasing the probability of tumour cure, without increasing the risk of normal tissue morbidity. At present, there are no effective biological response modifiers for the treatment of radiation-induced normal tissue lesions, including those of skin, mucus membranes or the CNS.

The present inventor has evaluated a number of herbal extracts in the search for a suitable biological response modifier. These substances, which demonstrated a limited success, included oral administration of the extracts of Chinese anti-inflammatory herbs Scutellaria barbata, Paeonia lactiflora, Salvia miltiorrhiza, E zhu, Glycyrrhica uralensis, Astragalus membranaceus, Lonicera japonica, Paeonia sufruticosa, and Trichosanthes kirilowii. These extracts had no beneficial effect in controlling the incidence of radiation-induced moist desquamation of skin in a rat model but accelerated its healing (Rezvani et al., unpublished data). In another study the efficacy of extracts of Fagopyrum esculentum, Symphytum officinalis and Calendula officinalis, given orally, had no beneficial effect in controlling the incidence of radiation-induced moist desquamation. It appeared that these herbal extracts also accelerated the healing of the moist desquamation (Rezvani et al., unpublished data).

There is no literature suggesting the involvement of sun flower oil in the treatment of radiation-induced lesions. However, Hopewell, et al., [(1994), New approaches to cancer therapy: unsaturated lipids and photodynamic therapy. Ed. D. F. Hobrobin, Churchill Communications, Europe (London) pp 88-106] reported that using sun flower oil as a placebo had some beneficial effects in modification of radiation-induced skin lesions in pigs.

α-Tocopherol (vitamin E) has not been used on its own in the treatment of radiation lesions but has been used as part of a combination treatment, administered for 8 weeks, 24 hours after irradiation of the skin of rabbits and showed no beneficial effect [Lefaix, et al., (1992), Bull. Cancer/Radiother. 79:189-198]. However, α-tocopherol in combination with Pentoxyfilline was significantly effective in softening and shrinking of radiation-induced fibrotic scar in pig skin [Lefaix, et al., (1999), Int. J. Radiat Oncol. Biol. Phys. 43: 839-847] and human [Delanian, S., (1998) British Journal of Radiology 71:.892-894].

Curcumin (diferuloyl methane) is a phenolic antioxidant and anti-inflammatory available in the rhizome of the plant Curcuma longa Linn. (Zingiberaceae). This yellow phytochemical can be extracted from this plant with ethanol or other organic solvents. Curcumin has strong antioxidant and free radical-scavenging activity and inhibits lipid peroxidation, including radiation-induced lipid peroxidation. Its anti-inflammatory action may be due to its inhibitory effect on arachidonic acid metabolism via the lipoxygenase and cyclooxygnase pathways [Stoner & Muditar, (1995) J. Cell. Biochem. Suppl. 22: 169-180].

Curcumin is involved with Hemox-1, and protects endothelial cells [Motterlini, et al., (2000) Free Radio. Biol. Med. 25:1303-1312] and it is a potent inhibitor of mutagenesis and has demonstrated strong anticancer activity [Huang et al., (1988) Cancer Res. 48: 5941-5946; Mehta R. G., and Moon R. C. (1991) Anticancer Res. 11: 593-596; Nagabhushan M., and Bhide S. V. (1992) J. Am. coll. Nutr. 11:192-198; Rao, et al., (1993) Carcinogenesis 14: 2219-2225; Huang, et al., (1994) Cancer Res 54: 5841-5847; Lu, et al., (1994) Carcinogenesis 15: 2363-2370; Subramanian, et al., (1994) Mutation Res. 311:249-255; Ramachandran C. and You W. (1999) Breast Cancer Research and Treatment 54: 269-278; and Inano H. and Onoda M. (2002) Int. J. Radiat. Oncology Biol. Phys. 52: 212-223].

Furthermore, reports indicate that curcumin inhibits the expression of c-fos, c-jun and c-myc proto-oncogenes [Rao et al., supra; Huang et al., supra; Lu et al., supra; Subramanian supra; Chen Y-R., Tan T-H. (1998) Oncogene 17: 173-178]. The biological and pharmacological properties of curcumin have been reviewed [Govindarajan V. S. (1980) CRC Rev. Food Sci. Nutr. 12: 199-301; Tonnesen H. H. (1988). Chemistry, stability and analysis of curcumin, a naturally occurring drug molecule. Ph.D. Dissertation, The Institute of Pharmacy, University of Oslo; Ammon, H. P. T., and Wahl, M. A., (1990) Planta. Med. 57: 1-7; Huang, et al., (1992) American Chemical Society, pp 339-349].

Curcumin, besides its antioxidant activities, has been shown to inhibit radiation induced protein kinase C activity [Varadkar, et al., (2001) J. Radiol. Prot. 21: 361-370]. Protein kinase C inhibits the ceramide pathway which in turn inhibits apoptosis. Curcumin can potentially interfere with this pathway (Varadkar et al, supra) and may cause tissue sensitisation.

The majority of the reports on curcumin involve its anticancer activities [Inano and Onoda, 2002, supra; Ramachandran and You, 1999, supra; Araujo, et al., (1999) Teratogen., Carcinogen., and Mutagen. 19:9-18]. There are no reports of the application of curcumin for the treatment of radiation induced skin or oral mucosal lesions, however. Administration of curcumin for two weeks prior to irradiation has successfully modified the radiation response assessed by the measurement of the glyoxalase activity in the liver and spleen of irradiated mice [Choudlary, et al., (1999) J, Ethnopharmacol. 64: 1-7] and it has been shown to be effective in the repair of both oxidative and reductive damages to proteins and oxidized amino acids caused by radiation [Kapoor S., and Priyadarsini K. I. (2001) Biophysical Chem. 92: 119-126].

WO 01/12130 discloses the use of curcumin to moderate the effects of glycolic acid in the treatment of scar tissue.

US 2001/0051184 discloses the use of curcumin to inhibit phosphorylase kinase and thereby treat inflammatory conditions. While the curcumin may be administered with other substances, such as antioxidants, it is essential for the curcumin to be dissolved in an alcoholic solution. Both water and mineral oils are stated to be completely ineffectual for dissolving curcumin and acting as carriers therefor.

It has now surprisingly been found that extracts of turmeric, in combination with edible oil and an antioxidant have beneficial effects in preventing and treating tissue damage caused by non-physical insult, such as chemotherapy and radiotherapy.

Thus, in a first aspect of the present invention, there is provided the use of a combination of curcumin, at least one antioxidant and at least one edible oil in the prevention and/or treatment of tissue damage caused by non-physical insult.

In a preferred aspect, the curcumin, antioxidant and edible oil are provided in combination, with the curcumin and antioxidant being at least partially, and preferably substantially completely dissolved in the oil, although it is possible to administer the components separately, if desired.

In a further aspect, there is provided the use of a combination of curcumin, at least one antioxidant and at least one edible oil in the manufacture of a medicament for the prevention and/or treatment of tissue damage caused by non-physical insult.

The medicaments of the present invention are useful as biological response modifiers, and may assist in repairing and preventing tissue damage in the course of treating cancerous growths, for example. It is common to provide non-physical insults to treat tumours and, while it is not intended to use the preparations of the present invention to compensate for physical insults, such as surgery, they may be used in conjunction with such treatments. In general, non-physical insults include chemical and radiation treatments, which are commonly employed to target rapidly growing tissues. The preparations of the present invention appear to allow greater levels of insult to be used, while minimising the effect on healthy tissue.

Particularly preferred insults are those arising from chemotherapy and radiotherapy, and especially ionising radiation.

As noted above, curcumin is diferuloylmethane and is present in the plant turmeric, Curcuma longa Linn. (Zingiberaceae). Accordingly, in one embodiment, the curcumin used in the present invention is in the form of an extract of turmeric. In general, it is preferred to use a more pure form of curcumin when making up any medication, in order that levels of purity and sterility may be controlled, although such considerations are not necessarily particularly important except when the medication is intended for injection, as extracts of turmeric are generally provided in a form suitable for ingestion.

Curcumin is abundantly available in oriental diet, for example, and it is on the FDA GRAS (generally recognised as safe) list. No LD₅₀ has been reported for it. Doses as high as 500-5000 mg/kg body weight have shown no toxicity when fed to animals (rats, cats, dogs, pigs and monkeys) over a period of 60 weeks.

Curcumin is metabolised in the gut to substances such as the glucuronides of tetrahydrocurcumin, hexahydrocurcumin, dihydroferulic acid and ferulic acid. Accordingly, the present invention also envisages use of the metabolites of curcumin, especially those identified above, in place of, or in addition to curcumin. It will be appreciated that curcumin may be used in the essentially pure form in the preparations of the invention, or may be provided in a form suitable for handling, such as pre-prepared in a small amount of oil for example. Metabolic precursors, such as ethers, for example, may also be employed instead of, or together with, curcumin in the preparation of the invention.

The curcumin is preferably provided in an oil. The oil is preferably a natural plant oil, more preferably a vegetable oil and most preferably sunflower oil. Other oils that may be used, either in combination with sunflower oil or in their own right, are linseed, olive, groundnut borage seed oil, hempseed oil, grape seed oil, walnut oil, wheat germ oil, soybean oil, corn oil, borage, evening primrose and rape (canola), with rape, borage, evening primrose and hempseed oils being the most preferred.

The level of curcumin in the oil will generally be determined by the skilled physician, but typically the percentage by weight of curcumin in the oil can be from 0.1%-60%, say from 0.5 to 25%, or about 1 to 20% w/v being convenient.

Preparations of the present invention comprise an antioxidant, and it has been found that vitamin E, or α-tocopherol, is particularly beneficial. Other antioxidants that may conveniently be used in the present invention include dimethyl sulfoxide (DMSO), gamma linolenic acid (GLA), melatonin, thiol-containing agents such as glutathione, methionine, cysteine, enzymes such as superoxide dismutase, catalase and glutathione peroxidase, ascorbic acid, selenium, carotene, flavonoids and extracts of plants such as Astragalus membraneus, Gingko biloba, Silybum marianum, Ligusticum chuaxiong, Panax ginseng and Scutellaria baicalensis. The preferred antioxidant is α-tocopherol, but any antioxidant suitable for administration in therapeutic amounts and preferably which is effectively non-toxic even in large quantities may also be particularly useful.

The ratio of curcumin: antioxidant, such as α-tocopherol, is suitably in the range of 25:1, to 2.5:1, more preferably 20:1 to 5:1, most preferably about 10:1. Where α-tocopherol is specified herein, it will be appreciated that this also includes any other antioxidant useful in the invention, unless otherwise apparent.

In a particularly preferred embodiment, the present invention provides a combination preparation comprising curcumin and α-tocopherol in an oil, especially sunflower oil. It is believed that sunflower oil itself makes a significant contribution to the healing effects of the product.

The efficacy of the preferred preparation is reflected in its remarkable DMF value of 1.44. Furthermore, all components of this, and other preferred embodiments of the invention, are non-toxic and can be administered in large quantities without fear of side effects.

It is preferred to use the preparations of the invention for the treatment of any tissue damage occurring through insults such as radiation. This can as simple as sunburn, or may be used to treat irradiated areas of skin immediately after treatment, for example. In any cancer treatment where tissue damage is observed, or likely, then administration of the preparations of the invention, whether as one or more components or together, is indicated.

Other suitable and preferred indications include protection of the CNS, especially for therapy near the spine, and treatment for mucositis for patients undergoing head and neck therapy, for example.

The preparations of the invention may be applied as a cream topically to an area of skin, mucus membranes or other tissues which have been exposed to radiation. Forms for topical administration include a mucus binding solution for oral mucosa, and a pessary for rectal administration. Alternatively, a formulation of the product suited for oral administration maybe provided, notably capsules or tablets containing the product, or the product may be administered in the form of a linctus directly per os, or by gavage, for example. Typically the amount of product taken daily by oral administration may be as low as 0.01 g per Kg bodyweight, but may is preferably 0.1 g-20 g per Kg body weight, say 0.25 to 10 g/Kg, or about 0.5 to 5 g/Kg.

The product of the present invention may be used for people who have been accidentally or unintentionally exposed to radiation, but it is mainly expected to be of use for patients receiving radiation therapy. A specific application for the invention is therapy-induced mucositis, notably mucositis induced by radiotherapy and/or chemotherapy. A particular example involves mucositis caused by radiotherapy of the, head and/or neck. Mucositis caused by conditions other than exposure to therapy, can also be treated by the present invention.

The invention also includes methods for the prevention and/or treatment of tissue damage caused by non-physical insult, comprising the administration to a patient in need thereof of a combination of curcumin, at least one antioxidant and at least one edible oil.

It is possible to separately administer the curcumin and α-tocopherol, but patient compliance is likely to be much better if a combined product of this invention is provided for administration.

Another method of this invention includes providing radiotherapy to a patient and administering a preparation of the invention. The radiotherapy can be as a single dose of radiation but will typically involve sequential exposure to doses of radiation, and the product of this invention is administered either before or after first exposure to radiation and continued after the completion of radiotherapy. In a preferred method, the product of this invention is administered after first exposure to radiation and continued after the completion of radiotherapy.

Typically the product of this invention is administered at repeated intervals following the end of exposure to radiation and, for example, the product will be given for at least 14 or, better, 28 days or more, following the last exposure to radiation.

The invention also includes treatment of mucositis and other conditions, including lesions in skin, the central nervous system (spinal chord and brain) or the gastrointestinal system. These other conditions may be induced by radiation. Examples of mucositis which can be treated include mucositis arising from exposure to radiotherapy, but the invention is not limited thereto, and conditions such as Irritable Bowel Syndrome can be treated. Thus, further aspects of this invention reside in methods of treating mucositis and other conditions with curcumin and α-tocopherol, compositions of curcumin and α-tocopherol for the treatment of mucositis and other conditions, and the use of curcumin and α-tocopherol in the preparation of medicaments for treating mucositis and other conditions. The methods of treatment extend to prophylactic treatments.

Further indications for preparations of the present invention include:

1—prevention of cancer, particularly development of radiotherapy-induced secondary tumours. 2—prevention and treatment of UV induced dermatitis (sunburn). 3—treatment of acute and chronic wounds on skin or buccal/oral mucosa and acceleration of healing of such wounds.

The invention embraces such methods, compositions for such methods, and the use of curcumin and/or α-tocopherol in the preparation of compositions for use in the methods.

A preferred form of sunflower oil is commercially available as Floral® pure sunflower oil with Vitamin E.

### EXAMPLE 1

### Amelioration of Radiation-Induced Normal Tissue Reactions

Amelioration of Radiation Induced Skin Lesions by Extract of Curcuma longa:

As noted above, the absence of cell production results in moist desquamation. The development of radiation-induced moist desquamation of rat foot is an established model for the study of this lesion [Rezvani, et al., (2002) B. J. R., 75: 50-55].

Mature (26 weeks old) female Sprague-Dawley rats were housed in groups of three per cage and received standard pellet diet food and water ad libitum. Both hind feet of each rat were irradiated, under anaesthesia, with a range of doses of ⁶⁰Co gamma rays, at a dose-rate of ˜1.3 Gy/min. Initially, animals were anaesthetised in a perspex box, flushed with oxygen and 2-3% halothane. Pre-anaesthetised rats were then positioned in a perspex irradiation jig and anaesthesia was maintained by continuous flushing with oxygen and 1-1.5% halothane at a rate of 2 l/min. The foot to be irradiated was positioned into a slot in a circular perspex holder (1 cm thick 11 cm diameter) located at the centre of the jig. Rats were positioned radially around this central perspex portion of the jig and nine animals were irradiated at each time. Irradiation schedules involved graded single doses of ⁶⁰Co γ rays to the left foot of the animal. The animals were then randomised into two groups of “test” and “placebo”.

The animals in the test group received 1 ml/day of an ethanolic extract of Curcuma longa (1:4, 25%) by oral gavage. Those in the placebo group received a similar volume of 25% ethanol in water by oral gavage. The feet were examined for the appearance of moist desquamation, daily, between 7 and 23 days after irradiation Nine animals were used per each dose point. Quantal data for the incidence of moist desquamation were analysed using logit analysis to provide ED₅₀ (±SE) values, the dose required to produce moist desquamation in 50% of irradiated feet, for both the test and the placebo groups. Dose modification factor (DMF) was obtained by dividing the ED₅₀ value for the test group by that of the placebo group. Data analysis was carried out by the SAS statistical package (SAS, 1989).

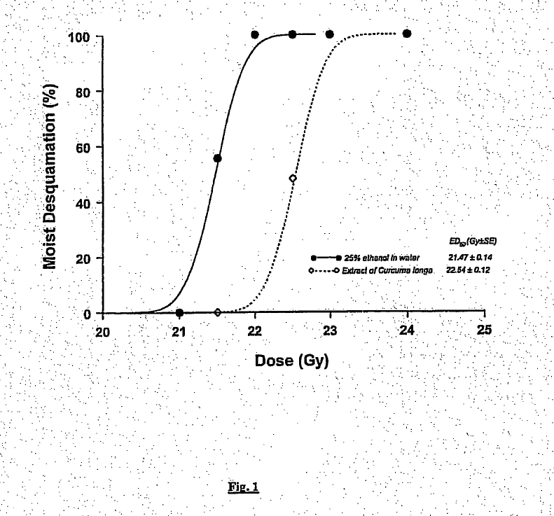

FIG. 1 shows the dose effect curves for the incidence of moist desquamation of rat foot skin after irradiation with graded doses of ⁶⁰Co γ rays for test and placebo groups. The ED₅₀ value of 22.54 Gy, for the incidence of moist desquamation in the skin of rat foot in animals treated with Curcuma longa extract, was significantly (p <0.01) higher than the value of 21.47 Gy, the ED₅₀ for the incidence of moist desquamation in placebo group. This difference in ED₅₀ values resulted in a DMF of 1.05.

Thus, although the effect was relatively small, the modifying effect of an extract of Curcuma longa was significant, at 5%, and is the first time that the incidence of radiation-induced moist desquamation in this model has been influenced by an exogenous factor.

### EXAMPLE 2

### Amelioration of Radiation-Induced Mucositis

A combination of curcumin (the active ingredient of the extract of Curcuma longa) and α-tocopherol, dissolved in sun flower oil, was tested. This compound demonstrated a significant beneficial effect in reducing the incidence of radiation-induced skin lesions and oral mucositis. The sun flower oil used appeared to have an additional effect, so that Preparation A, consisting of Curcumin, α-tocopherol and sun flower oil (SFO) was developed and tested.

Component of Preparation A:

Preparation A, as used in these Examples, consists of curcumin, α-tocopherol and sunflower oil. The dosage used in these Examples was α-tocopherol 20 mg/kg/day, curcumin 200 mg/kg/day in 0.5 ml/day SFO.

Although it is equally possible to use an extract of Curcuma longa, it was decided to use, as active ingredient curcumin (diferuloylmethane) which is the commercially, available active substance of the extract from Sigma-Adrich. Thus, the following Example used curcumin. α-Tocopherol (5,7,8-trimethyltocol) was also used. As the latter was not water soluble, both were solubilised in sun flower oil. Therefore, in some studies (results not shown) sun flower oil (SFO) was used as placebo. However, when sun flower oil treated animals were compared with those that received water (as placebo), or no drug treatment at all, it was found that sun flower oil itself had a beneficial effect in the treatment of radiation-induced oral mucositis and its addition enhanced the beneficial effects of curcumin and α-tocopherol.

Mature (12 weeks old; 200-225 g) female Sprague-Dawley rats were housed in groups of three per cage in conventional housing conditions, 55% humidity, 70-72°F, 12 to 12 hrs light-dark-cycle and received a standard pellet diet and water ad libitum. The animals were maintained, and all experimental procedures performed in compliance with the Animal (Scientific Procedures) Act 1986. While under anaesthesia the animal's tongue was slightly extended outside and a region of the underside of the tongue was irradiated in situ with single doses of 2.27 MeV β-rays from a 5 mm or 11 mm diameter ⁹⁰Sr/⁹⁰Y plaque. The dose-rate of the 5 mm ⁹⁰Sr/⁹⁰Y plaque was ˜10 Gy/min and that of 11 mm source was ˜3 Gy/min at the surface of the mucus membrane 5 mm source was used for single dose study and 11 mm source was used for fractionated studies. The irradiation was carried out by simply positioning the sealed ⁹⁰Sr/⁹⁰Y plaque in contact with the surface of the tongue. The tongue was stretched gently and radioactive source was placed with a uniform pressure in all cases in order to avoid any local hypoxia. In the unlikely event of local ischaemia/hypoxia occurring due to stretching the tongue or pressure of the source, the effect was ignored, as this would have equally applied for both control and test animals. The irradiation site was medial to the sublingual veins and a 4 mm margin was maintained from the tip of the tongue. Irradiation was carried out under general anaesthesia maintained with a Halothane/oxygen mixture.

Single Dose Studies:

A total of 144 rats were used for this part of study. Four groups of36 animals were irradiated with single doses of either 13.5, 15, 16.5 or 18 Gy. Following irradiation the animals in each dose group were subdivided into four treatment subgroups of 9 rats to receive 0.5 ml per day of either Preparation A, SFO, α-tocopherol or water by oral gavage until the end of experiments. Nine animals were used at each dose point in each treatment group. Mucosal ulceration (erosion of mucosal epithelium) was considered as an end-point and this is referred by radiation-induced mucositis in the context of present experiments. From the day after irradiation, until any acute radiation-induced oral mucosal lesion had healed, the animals' tongues were assessed daily for the presence of radiation-induced mucositis (mucosal ulceration) under light anaesthesia, maintained with a 1.5% Halothane, oxygen mixture. Daily assessment of mucositis was carried out under ×2 magnifying glass with a cold light prior to oral gavaging. Quantal data for the incidence of radiation-induced mucositis were analysed using logit analysis to provide ED₅₀ (±SE) values, the dose at which radiation mucositis (mucosal ulceration) was observed in 50% of irradiated tongues. The dose modification factor (DMF), defined as the ratio of the dose of radiation to cause mucositis in ‘active’ agent group to that in ‘placebo’ group, was calculated. Data analysis was carried out using SAS statistical package (SAS, 1989).

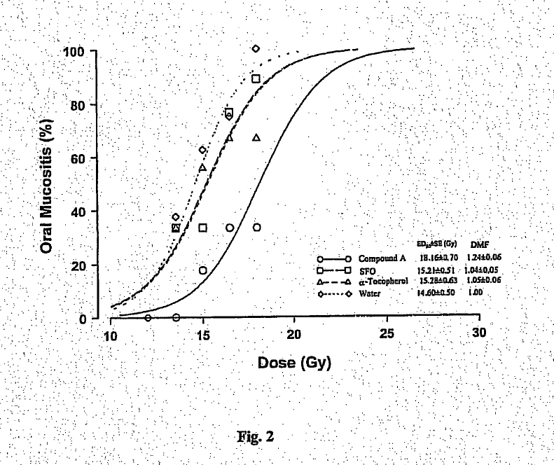

The incidence of radiation-induced oral mucositis (mucosal ulceration) in the tongue of rats is shown in FIG. 2. There was a modest increase in the ED50 values after both α-tocopherol and SFO administration that resulted in DMF values of 1.05 and 1.04, respectively. The ED₅₀ value of 18.16±0.70 Gy obtained for the treatment of radiation-induced moist desquamation after treatment with Preparation A was significantly (p<0.01) higher than that of the animals that received water, SFO or α-tocopherol.

Both α-tocopherol and SFO showed a modest beneficial effect in the treatment of radiation induced oral mucositis. However, Preparation A significantly reduced the incidence of radiation mucositis with a significant DMF value of 1.24±0.06.

Mucosal ulceration (erosion of mucosal epithelium) was considered as an endpoint and it appeared to be a reliable representation of radiation-induced oral mucositis. A similar end point has been used by other authors (Doerr et al., 2001, supra). The model involves the irradiation of only a small area of the underside of the tongue. This appears to be a useful model to study the clinically relevant endpoint of oral mucositis following irradiation. While a good dose-response relationship was obtained for the incidence of mucositis the reaction had no apparent effect on the animal's general well-being. There were no noticeable changes observed in animals with respect to body weight, eating, drinking or behaviour.

While the intensity and duration of mucositis are dose dependent, the latent period for the development of mucositis primarily depends on the turnover of the epithelial layers. In the present model the lesions developed from around 10 days after irradiation and the latency period was independent of the radiation dose and the treatment group.

### EXAMPLE 3

### Fractionated Dose Studies

Fractionated radiotherapy is employed in curative treatment. Accordingly, it was decided to assess Preparation A in relation to fractionated schedules. Normal fractionated radiotherapy consists of 25 fractions of 2 Gy delivered daily, 5 days per week. Such a schedule will be completed in 33 days. In a rat model, where radiation induced mucositis develops from around 10 days after irradiation, 25 fraction will span over the period of the development of mucositis. A schedule involving a short overall treatment time was required in this model. The most appropriate and established technique, which closely mimics clinical practice, consists of a limited number of 2 Gy fractions followed by a large top-up dose.

A total of 126 rats were used for this Example. Three groups of 36 and one group of 18 animals were irradiated with eight daily fractions of 2 Gy (5 fractions per week) followed by single top-up doses, of various sizes (7.5-17.5 Gy). The first fraction was always started on a Monday and top-up dose was delivered on Thursday of the following week. Following irradiation, the animals in each dose group were subdivided into four treatment subgroups. Nine animals were used at each dose point in each treatment group. Group 1 (Radiation only) received no further treatment except radiation. There were 36 (4×9) animals in this group. Group 2 (water) received 0.5 ml per day of water by oral gavage. There were only 18 (2×9) animals in this group. Group 3 (SFO) received 0.5 ml per day of sunflower oil. Group 4 (Preparation A) received 0.5 ml per day of Preparation A. Tested substances and placebo (water) were administered daily by oral gavage starting after the first 2 Gy fraction and continued until the end of experiments. Mucosal ulceration (erosion of mucosal epithelium) was considered as an end-point and this is referred by radiation-induced mucositis in the context of present Examples. From the day after start of irradiation until any acute radiation-induced oral mucosal lesion had healed, the animals' tongues were assessed daily for the presence of radiation-induced mucositis (mucosal ulceration) under light anaesthesia, maintained with a 1.5% Halothane, oxygen mixture.

Daily assessment of mucositis was carried out under ×2 magnifying glass with a cold light prior to oral gavaging. Quantal data for the incidence of radiation-induced mucositis were analysed using logit analysis and top-up ED₅₀ (±SE) values, the top-up dose at which radiation mucositis (mucosal ulceration) was observed in 50% of irradiated tongues were obtained. The dose modification factor (DMF) defined as the ratio of the top-up dose of radiation to cause mucositis in test group to that in radiation only group was calculated. Data analysis was carried out using SAS statistical package (SAS, 1989).

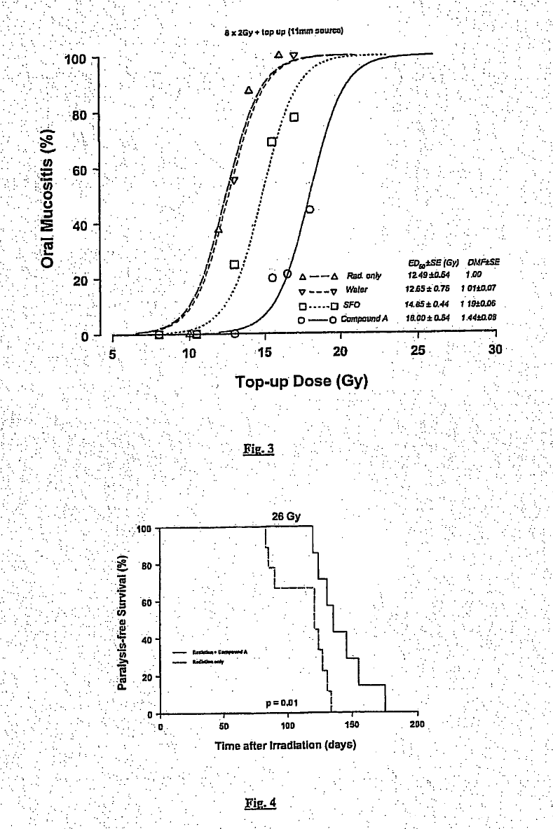

The incidence of radiation-induced oral mucositis (mucosal ulceration) in the tongue of rats after fractionated irradiations are shown in FIG. 3. There was no significant difference in the incidence of radiation-induced mucositis in water treated (placebo) annuals and radiation only group. However, the ED₅₀ values of 14.85±0.44 Gy and 18.00±0.08 Gy for the incidence of radiation-induced mucositis in SFO and Preparation A groups, respectively were significantly higher than that of the radiation only and placebo groups. This resulted in significant DMF values of 1.19±0.06 and 1.44±0.08, respectively. FIG. 3 shows the incidence of radiation-induced mucositis in rats after irradiation with eight daily fractions of 2 Gy (5 fractions per week) followed by a single top-up dose. Total dose=8×2 Gy+Top-up dose. Test substances, Preparation A and SFO, and placebo (water) were given in 0.5 ml volumes starting after the first 2 Gy per fraction and continued until the end of experiments.

The fractionated studies support the results of the single dose study of Example 2, and further reveal the beneficial effect of both Preparation A and SFO in the treatment of radiation-induced oral mucositis. The effect of both substances is enhanced after fractionated irradiations, apparent in the DMF values of 1.44 and 1.19, respectively.

### EXAMPLE 4

### Comparison of the Efficacy of the Components of Preparation A

A total of 36 rats were used for this Example. All animals were irradiated with eight daily fractions of 2 Gy (5 fractions per week) followed by single top-up of 16.5 Gy. The first fraction was always started on a Monday and top-up dose was delivered on Thursday of the following week. Following irradiation the animals were subdivided into four treatment groups of nine rats. Group 1 (preparation A) received 0.5 ml per day of Preparation A. Group 2 (SFO+Curc.) received 200 mg/kg/day Curcumin in 0.5 ml SFO. Group 3 (SFO+Toco.) received 20 mg/kg/day α-tocopherol in 0.5 ml SFO. Group 4 (Toco+Curc.) received 20 mg/kg/day α-tocopherol and 200 mg/kg/day Curcumin in 0.5 ml water. Tested substances were administered daily by oral gavage starting after the first 2 Gy fraction and continued until the end of experiments. Mucosal ulceration (erosion of mucosal epithelium) was considered as an end-point and this is referred to as radiation-induced mucositis in the context of present experiments. From the day after start of irradiation until any acute radiation-induced oral mucosal lesion had healed the animals' tongues were assessed daily for the presence of radiation-induced mucositis (mucosal ulceration) under light anaesthesia, maintained with a 1.5% Halothane, oxygen mixture. Daily assessment of mucositis was carried out under ×2 magnifying glass with a cold light prior to oral gavaging.

Table 1, below, shows the effect of Preparation A and its components on the incidence of radiation-induced oral mucositis in rats irradiated with 8×2 Gy daily fractions followed by a top-up dose of 16.5 Gy. Different combinations of the components of Preparation A show a degree of effect in reducing the incidence of radiation-induced oral mucositis, but the greatest effect is produced by Preparation A itself containing all components.

TABLE 1
Influence of Preparation A and its components on the incidence of
radiation-induced oral mucositis in rats irradiated with 8x2Gy daily
fractions followed by a top-up dose of 16.5 Gy.

| | Incidence of |
|---|---|
| | oral mucositis (%) |
| Modifying agent | (responder/at risk) |
| Preparation A | 11.11% (1/9) |
| SFO + Curcumin | 66.67% (6/9) |
| SFO + α-tocopherol | 33.33% (3/9) |
| α-tocopherol + Curcumin | 55.56% (5/9) |

Fischer's exact test revealed that the incidence rates shown in Table 1 were significantly different (p<0.05). This implies that all three components of Preparation A are making contribution in alleviating the incidence of radiation-induced oral mucositis in rat.

### EXAMPLE 5

### Modification of Radiation Myelopathy in the Rat

Five week old female Sprague-Dawley rats, 90±10 g, were housed in groups of four per cage and received a standard pellet diet and water ad libitum. A 12 mm length of the cervico-thoracical cord (T2-C2) was irradiated, under halothane anaesthesia, with 26 Gy single dose of 250 kV X-rays, at a dose-rate of ˜1.4 Gy/min. For irradiation, pre-anaesthetised rats (2-3% halothane in oxygen) were restrained in the jig and the position of the large thoracic vertebral spine was identified by X-ray fluoroscopy. A perspex collar, 10 mm thick, and specifically shaped to fit the neck of the animal, was then positioned to restrain the animal. This collar contained two lead wire markers 12 mm apart. The caudal marker was placed over T2 and the area between the two makers was identified as the length of spinal column to be irradiated. The rest of the animal was shielded by a 4 mm thick lead with a 12 mm cut-off guided again by the lead wire markers. During irradiation, anaesthesia was maintained continuous flushing of the irradiation jig with oxygen and 1.5% halothane at a rate of 2-3 l/min.

Two animals were irradiated at a time. One was allocated to the control group and the other to the test group, after irradiation. A total of 18 animals were irradiated; 9 in the control and 9 in the test groups. A single dose of 26 Gy, as employed in this Example, produces 100% myelopathy within five months of irradiation, in this model.

After irradiation, the animals in the test group received a daily dose of 0.5 ml of Preparation A, as prepared in Example 2 above, by oral gavage. Oral gavaging was carried out for 8 weeks starting from the first day of irradiation. The first dose was given immediately after irradiation. The animals in the control group received only the radiation and no Preparation A.

Data analysis was performed using the SAS statistical package. Non-parametric life table estimates were calculated and log-rank test was carried out to identify the association of the survival time with treatment group.

The paralysis-free survival rates for rats, after irradiation with 26 Gy of 250 kV X-rays, in both the control and the active groups are shown in accompanying FIG. 4. In FIG. 4, the curves show paralysis-free survival times after 26 Gy to a 12 mm length of cervical spinal cord, the dashed line being for the radiation only (‘control’) animals, and the solid line being for those treated with Preparation A for 8 weeks after irradiation. The P- value relates to the log-rank test.

Animals receiving only radiation all developed paralysis within no more than 134 days of irradiation. The mean latency (±SE) period for the development of paralysis in this group of animals was 112.9±6.9 days (83-134 days).

By the time that all of the control animals exhibited paralysis, over 50% of the animals in the test group were paralysis-free. The animals of the test group had all developed paralysis within 174 days of irradiation. The mean latency (±SE) period for the development of paralysis in this group of animals was 141±7.3 days (120-175 days); 26% longer survival.

However, it should be noted that the administration of Preparation A was only for 8 weeks after irradiation. Nevertheless, the latency period for the incidence of radiation induced myelopathy was significantly prolonged (p=0.02). Overall, the difference between paralysis-free survival of control and drug treated rats was statistically significant (p=0.01).

1. The use of a combination of curcumin, or precursor or derivative thereof, at least one antioxidant and at least one edible oil in the prevention and/or treatment of tissue damage caused by non-physical insult.

2. Use according to claim 1, wherein the curcumin, antioxidant and edible oil are provided in combination, with the curcumin and antioxidant being at least partially dissolved in the oil.

3. Use according to claim 2, wherein the curcumin, antioxidant and edible oil are provided in combination, with the curcumin and antioxidant being substantially completely dissolved in the oil.

4. The use of a combination of curcumin, at least one antioxidant and at least one edible oil in the manufacture of a medicament for the prevention and/or treatment of tissue damage caused by non-physical insult.

5. Use according to any preceding claim, wherein the curcumin is provided in the form of rhizomal extract of Curcumin longa Linn.

6. Use according to preceding claim, wherein diferuloyl methane is used as the curcumin component.

7. Use according to any preceding claim, wherein the insult is selected from chemotherapy and radiotherapy.

8. Use according to any preceding claim wherein the tissue damage is mucositis.

9. Use according to any of claims 1 to 7, wherein the tissue damage is to the central nervous system.

10. Use according to any preceding claim wherein the oil is a vegetable oil.

11. Use according to claim 10, wherein the oil is selected from at least one of the group consisting of linseed oil, olive oil, groundnut oil, borage seed oil, hempseed oil, grape seed oil, walnut oil, wheat germ oil, soybean oil, corn oil, borage seed oil, evening primrose oil and rapeseed oil.

12. Use according to claim 10 wherein the oil is sunflower oil.

13. Use according to any preceding claim, wherein the antioxidant is selected from at least one of the group consisting of include α-tocopherol, dimethyl sulfoxide, gamma linolenic acid, melatonin, thiol-containing agents, enzymes, ascorbic acid, selenium, carotene, flavonoids and extracts of Astragalus membraneus, Gingko biloba, Silybum marianum, Ligusticum chuaxiong, Panax ginseng and Scutellaria baicalensis.

14. Use according to claim 14, wherein the antioxidant is α-tocopherol.

15. Use according to any preceding claim, wherein the curcumin antioxidant and oil are formulated for co-administration.

16. Use according to any preceding claim, in the treatment of the effects of exposure to radiation.

17. A combination preparation as defined in any preceding claim, wherein the curcumin is present in an amount of 0.1 g-20 g per Kg body weight.

18. A formulation according to claim 17, in a dosage form suited for topical or oral administration.

19. A method for the prevention or treatment of tissue damage caused by non-physical insult, comprising the administration to a patient in need thereof of a combination of curcumin, or precursor or derivative thereof, at least one antioxidant and at least one edible oil, wherein said components or indications are in accordance with any of claims 1 to 16.

20. The method of claim 19, wherein the curcumin, or precursor or derivative thereof, at least one antioxidant and at least one edible oil are formulated and administered together.

21. The method of claim 19, wherein said combination is administered in conjunction with a course of radiotherapy.

22. The method of claim 19, wherein said combination is administered in conjunction with a course of chemotherapy.

23. The use of a combination of curcumin, or precursor or derivative thereof, at least one antioxidant and at least one edible oil, wherein the components are as defined in any of claims 1 to 16, for the:
prevention of cancer, particularly development of radiotherapy-induced secondary tumours.
prevention and treatment of UV induced dermatitis (sunburn).
treatment of acute and chronic wounds on skin or buccal/oral mucosa and acceleration of healing of such wounds.
